(19) United States
(12) Patent Application Publication
(10) Pub. No.: US 2014/0330159 A1
(43) Pub. Date: Nov. 6, 2014

(54) QUANTITATIVE METHODS AND SYSTEMS FOR NEUROLOGICAL ASSESSMENT

(71) Applicants: BETH ISRAEL DEACONESS MEDICAL CENTER, INC., Boston, MA (US); PRESIDENT AND FELLOWS OF HARVARD COLLEGE, CAMBRIDGE, MA (US)

(72) Inventors: Madalena Damasio Costa, Brookline, MA (US); Leia A. Stirling, Stoneham, MA (US); James B. Niemi, Maynard, MA (US); Ary L. Goldberger, Newton Centre, MA (US)

(73) Assignees: BETH ISRAEL DEACONESS MEDICAL CENTER, INC., Boston, MA (US); PRESIDENT AND FELLOWS OF HARVARD COLLEGE, CAMBRIDGE, MA (US)

(21) Appl. No.: 14/347,306

(22) PCT Filed: Sep. 26, 2012
(86) PCT No.: PCT/US12/57270

§ 371 Date: Mar. 26, 2014

Related U.S. Application Data
(60) Provisional application No. 61/539,409, filed on Sep. 26, 2011.

(51) Int. Cl. A61B 5/11; A61B 3/113; A61B 5/00

(52) U.S. Cl. CPC A61B 5/1124; A61B 5/40; A61B 5/7246; A61B 3/113; A61B 5/7475
USPC 600/558; 600/595

(57) ABSTRACT

Typical neurological examinations focus on qualitative and subjective assessments, including obtaining a patient history, assessing the patient's cognitive status, motor and sensory skills, and cranial nerve functionality. A quantitative assessment of neurological condition includes recording a subject performing a visuomotor task and processing the performance data to determine a level of complexity in the task activity and determine a complexity index. For a sample healthy population, a baseline level of complexity and baseline complexity index can be determined. A patient's complexity index can be compared to this baseline complexity index as an indication of disease or disability. A baseline complexity index can be determined for a patient at part of a health maintenance examination and used as the baseline complexity to detect disease or disability in the future based on lower complexity index values in future examinations.

### CROSS-REFERENCE TO RELATED APPLICATIONS

This application is related to U.S. Pat. Nos. 7,601,124 and 7,882,167, the entire contents of which are hereby incorporated by reference in its entirety.

This application claims any and all benefits as provided by law including benefit under 35 U.S.C. §119(e) of the U.S. Provisional Application No. 61/539,409, filed Sep. 26, 2011, the contents of which are incorporated herein by reference in its entirety

### STATEMENT REGARDING FEDERALLY SPONSORED RESEARCH

This invention was made with government support under grants no. U01-EB-008577 and AG030677 awarded by the National Institutes of Health. The government has certain rights in the invention.

### REFERENCE TO MICROFICHE APPENDIX

Not Applicable

### BACKGROUND

### Technical Field of the Invention

Typical neurological exams focus on several qualitative assessments, including obtaining a patient history, assessing the patient's cognitive status, motor and sensory skills, balance and coordination, reflexes, and functionality of the cranial nerves. Common motor assessments include examining for pronator drift, testing range of motion, examining muscle tone, and touching the thumb to the fingers in rapid succession. Most of these skills are rated on very general scales with course gradations, making assessment of change difficult and also subjective. For example, strength is graded as follows: 0—No movement, 1—flicker of movement, 2—able to move with gravity, 3—able to move weakly against gravity, 4—weak against resistance, 5—full strength against resistance. The same patient may be scored differently by two observers on the same occasion. In addition, assessment of progression over time may lack objectivity, sensitivity, and consistency. In the case of concussions, the assessment is performed on several parameters with the scale 0 (no symptoms)—5 (severe symptoms). Both of these examples illustrate the subjectivity with respect to the practitioner interpreting the scale and the patient's own interpretation of their symptoms.

Previous studies have shown that a simple visuomotor tracking task that is based on tracing a circle correlates with gross gait and posture measurements in those with Parkinson's disease (Inzelberg et al., 2008) and with changes in medication in those with attention deficit disorder (Tirosh et al., 2006) and Parkinson's disease (Hocherman et al., 1998). While this tracking task yields quantitative information, there is no dynamical analysis of the time series profiles.

Previous work has shown that clinically important information is encoded in the fluctuations of physiological time series (Lipsitz and Goldberger, 1992; Goldberger et al. 2002). These fluctuations represent the dynamics of the underlying control systems. For example, fine motor movement variability has been shown to increase with age, specifically with impairments in executive control (Krampe, 2002). However, traditional measures of variability (e.g. standard deviation, spectral power) may not fully characterize the structure of physiologic time series, as the physiological control mechanisms normally operate over many time scales (Goldberger, 1996). From biological and clinical points of view, complexity (in a subject's control of movements) is related to adaptability and integrative functionality (Costa et al., 2002; Costa et al. 2005). One computational tool developed to analyze complex-appearing signals is called multiscale entropy (MSE). This method quantifies the information content of a time series over a range of scales (Costa et al., 2002; Costa et al. 2005). Briefly, the method comprises three steps: 1) a coarse-graining procedure used to construct a set of derived time series representing the system's dynamics over a range of scales, 2) quantification of the degree of irregularity for each coarse-grained time series using an entropy measure, sample entropy and 3) calculation of the complexity index, CI. The sequence of entropy values for a range of scale is called the MSE curve. The method has been used to show that disease and aging lead to a loss or degradation of multiscale complexity, which in turn reflect system adaptability. For example, patients with congestive heart failure and atrial fibrillation show a marked reduction in heart interbeat interval complexity compared to healthy control subjects (Costa et al., 2002) and older adults have a reduction in balance complexity compared to younger adults (Costa et al., 2007). Major clinical depression in young to middle aged men also is associated with loss of heart rate complexity during sleeping hours (Leistedt et al, 2011).

Previous studies of human movement have presented that even continuous motions are composed of potentially many quantized sub-movements (Meyer et al., 1990; Krebs et al., 1999). These sub-movements were observable after stroke as isolated movement segments that became more overlapped with recovery (Krebs et al., 1999; Rohrer et al., 2002) and in babies prior to maturing their reaching strategies (von Hofsten, 1980).

### SUMMARY

The present invention is directed to method and systems for quantifying a neurological function by providing a neuromotor or visuomotor task and tracking a patient's performance of the task. The tracking system monitors the patient's ability to perform the task with physical accuracy and temporal accuracy, thus the system tracks both positional information and temporal information. Using the positional data and the temporal data, a neuromotor index, which can include a multiscale complexity analysis, can be used to assess the complexity or lack of complexity indicative of decreased neuromotor function. In addition, the same assessment can be used to determine, quantitatively, an increase or decrease in neuromotor function as an indicator of the onset of disease or to evaluate the effectiveness of treatment over extended periods of time. In accordance with some embodiments of the invention, additional or alternative components of the neuromotor index can include the cumulative micropause duration and percent time in the target region, Fourier decomposition indices, Tsallis entropy, Kolmogorov entropy, diffusion entropy, detrended fluctuation analyses, box counting analyses, wavelet analyses, Hilbert-Huang Transforms, and empirical mode decomposition.

One object of the present invention is to obtain a quantitative measure with sufficiently high resolution to provide clinically useful information on the subject's visuomotor ability and neuromotor functionality, for example, using a fully-automated recording and analysis system.

In accordance with one embodiment of the invention, a measure of submovement concatenation, the micropause timing of the participant can be recorded. A micropause can be defined as the time when the velocity was zero. Where the task (tracing a circle) involved a continuous motion, it can be inferred that pauses are primarily due to delays in concatenation of submovements and thus provide an indication of neurological performance.

The tradeoff between speed and accuracy in task performance has been well documented and was shown originally by Fitts (1954) that faster motions permit less error correction and thus have decreased accuracy as compared with slower motions. In accordance with one embodiment of the invention, “microcontrol adaptations” can be defined as the real-time error corrections that occur during a motion task. These microcontrol adaptations may be adjusted by feed-forward or feedback control mechanisms and rely on real-time sensory processing. In addition, the percent time in the target region can be used to evaluate the accuracy of the subject's imposed control method.

The implementation of complexity analyses with regard to tracking is not clear from the literature. Studies by Slifkin et al. (2000) and Newell et al. (2003) use a force-production tracking task to show that complexity is dependent on age and task. This concept is expanded in Vaillancourt and Newell (2002). Their argument is that tasks involving a fixed-point will have a higher complexity for healthy subjects and a lower complexity for age/disease, while a task with intrinsic dynamics (such as a sine curve) will have the opposite result. Goldberger et al. (2002) argue that the increases in complexity are due to inappropriate usage of the non-linear methods due to the breakdown of correlation properties, and alteration of nonlinear interactions.

In accordance with the invention, it is believed that the increase or decrease in complexity is related to how the task data are pre-processed and compared by the subject. Further, in accordance with some embodiments of the invention, the residual or difference signal (e.g., the difference between the recorded and desired force trajectory) can be analyzed and it is believed that this portion of the signal is directly related to the microcontrol corrections of the subject. An assessment of the subject can be made by comparing the residual signal to a baseline. Thus, the complexity of the task with intrinsic dynamics can be presented in a similar manner as the fixed-point task and can be comparable.

In accordance with one embodiment of the invention, a quantitative measure or index can be determined by preprocessing and complexity-based analysis of the micro-error data, using, for example, known functions for performing the complexity-based analysis from the prior art. In accordance with one embodiment of the invention, the complexity-based analysis can be performed on the residual or error data (e.g., micro-error data), and not on the raw signal. The micro-error data can be produced by monitoring a subject performing a physical, visuomotor task, such as following an object (e.g., a block or circle) along a path with their finger (or pointing device), and for each data point, determining the difference between the position of the finger and the location of the path to be followed in one, two or three dimensional space. Further, additional micro-error data can be determined as the difference between the time that the finger is expected to be at a particular location and the actual time it takes to get to a given location (or the closest position to that location), recognizing that the time value could be positive (e.g., delayed motion) and negative (e.g., arrived early). Each of these sets of micro-error data form a time series of data to which multiscale complexity analysis can be applied. For a range of scale factors, a set of entropy values can be plotted and the area under the plot can be determined and used to produce a complexity index that can be used as the neuromotor index or combined with other indices to form the neuromotor index.

In accordance with some embodiments of the invention, other data can be monitored and used in the assessment. For example, a representation of sub-movement aggregation can be determined by monitoring the cumulative micropause duration of the subject, which can be defined as the sum of the time durations when the velocity is zero. In a task that involves continuous motion, pauses in motion indicate delays in the concatenation of sub-movements and provides an indication of neurological performance or micropause index that can be used as the neuromotor index or combined with other indices form the neuromotor index. In accordance with some embodiments of the invention, the percent time in the target region can be used to evaluate the accuracy of the user's imposed control method. Similarly, we can include other metrics representing the phase of the micro-error with respect to the template task, including the definition of a new time series from the original micro-error data that includes the relative phase or position of the stylus with respect to the template, or the micro-error signal itself.

In accordance with some embodiments of the invention, the neuromotor index can be used as a measure of executive control.

To address the deficiencies in typical neurological assessment, various embodiments of the present invention can include a task tracking device or system that records the movements of a patient while the patient is performing a task, such as, following a pre-determined path (such as a circle, sine wave, noisy or random pattern, spiral, etc.) and computes one or more adaptability parameters. This device can be employed to provide assessments in many areas, described below.

In accordance with some embodiments of the present invention, the task tracking device can include a computer system or data processing system, including one or more processors and associated memory, a user input/output component (e.g. a monitor, a touch screen or a touch pad, a keyboard and mouse) and a task monitor. The task monitor can include one or more sensors that monitor a subject interacting with the device while performing one or more visuomotor tasks and provide data to the computer system. The computer system can control the operation of the task monitor and, at the same time, receive and store the data generated by the task monitor. The computer system can also process the data and produce additional data (e.g., micro-error data, micropause data, or percent in region data) or the raw data can be transferred to another data processing system to produce the additional data.

In one embodiment according to the invention, the tracking system can include one or more software modules or applications that can be used on a touchscreen sensitive tablet (e.g., a tablet computer, a tablet device such as an Apple iPad or Google Android based device, or an external drawing/touch sensitive surface that connects to a computer) that records the subject's position as they execute a defined task (e.g., follow an object along a defined or displayed path). The subject can trace the path using either a stylus (pen, etc.), pointing device, or their fingertip. On a touch screen, the path can be displayed along with an object that moves along the path at a predefined speed (or speed profile) and the subject can follow the object with their finger (or stylus) as it moves along the path. Where the touch sensitive surface does not also include a display, the path and the object can be printed, drawn or projected on to the surface.

In a second embodiment according to the invention, the tracking system can include a display (which could be a monitor or a projected image) and an eye-tracking system. In this embodiment, the path following task can be measured by examining the motion of the eyes using the eye tracking system. The eye tracking system can determine the location of gaze and the micro error data can be produced based on the difference between the position of the object along the path and the actual gaze of the eyes.

In a third embodiment according to the invention, the tracking system can employ a motion capture system to obtain the body motion. The body motion can be captured with methods including, but not limited to, cameras, accelerometers, gyroscopes, magnetometers, and force sensitive resistors.

In a fourth embodiment according to the invention, the tracking system can use a pointing laser to trace the motion of a moving target. The target can be moved by a simple mechanism, such as a rotating disk or a more complex system, multi degree-of-freedom actuator, such as a robotic arm. The target can include one or more sensors that can be used to determine the position of the laser image (laser spot) on the target and the deviation of the image from a center or reference point on the target. The position to be tracked could also be on a display screen.

In these embodiments, the motions of a subject that can be captured include, but are not limited to the head, eyes, arms, hands, legs, feet, torso, full body, or external tool.

Further, in accordance with the invention, the determination of dynamical complexity can include a tolerance component, such as a noise rejection level, that can be selected according to one or more predefined parameters. In accordance with one embodiment of the invention, the tolerance is determined as a function of the sampling period of data points collected.

One object of the invention is to provide a method and system for measuring a neuromotor tracking function in a way that is simple, accurate, quantitative and inexpensive. The system outputs can be measurements including what is referred to as the neuromotor index, an index that can probe one or more of the characteristics a physiologic system. These characteristics can include 1) correlations across multiple time scales, 2) accuracy, and 3) fluidity. These characteristics can be analogized to the properties that are universally understood to underlie great works of classical music. From a practical, clinical point of view, we can assess and report these characteristics using the neuromotor index by employing a number of methods designed to analyze series of data points (time series).

Correlations across multiple time scales and information content can be assessed with a variety of entropy-based analyses, including but not limited to: multiscale entropy (MSE), sample entropy (SampEn), approximate entropy (ApEn), Tsallis entropy, Kolmogorov entropy, and diffusion entropy.

Complementary techniques that measure correlation properties of a time series include those derived from the theory of chaotic systems and fractal and multifractal analyses. The former includes calculation of Lyapunov exponents and quantification of the degrees of freedom of a system. The latter focuses on calculating fractal exponents using techniques such as detrended fluctuation analyses, Hurst re-scaled range analysis, and wavelet-based methods. The multiscale entropy (MSE) method has certain attractive features for capturing correlations across time scales and information content in that it explicitly measures the entropy, not only of the original signal, but also of a family of signals derived therefrom, which represent multiple time scales. This technique allows one to distinguish highly variable signals without correlations (e.g., white noise) from more physiologic types of 1/f noise seen in the output of complex adaptive systems.

The accuracy of the tracking motions can be assessed with a number of measures in the time domain, including mean, standard deviation and higher moments of the histogram/probability distribution.

The fluidity of motion can be assessed by detecting oscillations that indicate the presence of a characteristic time scale associated with a pathologic process. For example, Parkinson's disease is associated with distinctive oscillations (tremor) at a frequency of around 5 Hz. These periodic dynamics can be detected and quantified using frequency domain analyses, including, for example, Fourier or wavelet-based methods, and methods based on the Hilbert-Huang Transform (HHT) and empirical mode decomposition (EMD). The assessment of fluidity (or lack thereof) of motion can also be assessed using measures of micropauses and reflected in the micropause index and as a component of the neuromotor index. The greater the number of pauses and the longer their duration, the less fluid the motions are.

These and other capabilities of the invention, along with the invention itself, will be more fully understood after a review of the following figures, detailed description, and claims.

### BRIEF DESCRIPTION OF THE FIGURES

FIG. 1 shows a diagram of a task tracking system according to an embodiment of the invention.

FIG. 2 shows a diagram of a task monitoring device according to an embodiment of the invention.

FIG. 3 shows a diagram of a task according to an embodiment of the invention.

FIG. 4 shows a diagram of residual or micro-errors according to alternative embodiments of the invention.

FIG. 5 shows a diagram of an example of a target path, an actual path and a residual signal according to an embodiment of the invention.

FIG. 6 shows a diagram of a target region according to an embodiment of the invention.

### DETAILED DESCRIPTION OF PREFERRED EMBODIMENTS

The present invention is directed to methods and systems for providing quantitative neurological assessments and neuro-diagnostic evaluations. In accordance with various embodiments of the invention, a subject is asked to perform a task that is intended to test a specific neurologic function and a system tracks and records the subject's performance of the task. The task data can be analyzed using a neuromotor index. This index can include a multiscale complexity analysis in order to determine a quantitative assessment, such as a Complexity Index (CI) which can be compared to a standard or baseline index for the subject to detect disease or disability, or the CI can be compared to prior performance data and CI values for the subject to assess effectiveness of treatment or therapy. The neuromotor index can also include timing parameters, such as the cumulative micropause duration, which is an indicator of motion submovement concatenation and percent time in target region. A further detailed description of the data processing is included below.

In accordance with embodiments of the present invention, mathematical methods derived from the theory of nonlinear systems can be used quantify the complexity of a signal derived from tracking a subject performing a task. The signal to be quantified can be one or more of the following: 1) the recorded trajectory; 2) the residual signal defined as the difference between the recorded trajectory and the actual (task target) trajectory; 3) any signal derived from each of two previously mentioned, such as those obtained by computing their first and second derivatives, representing velocity and acceleration, respectively. In accordance with the invention, the methods used to quantify the signal include, entropy-based algorithms such as multiscale entropy, sample entropy, approximate entropy, algorithms that quantify the fractal properties of a signal such as detrended fluctuation analysis, time domain parameters such as the moments of a distribution (mean, variance, skewness, etc.), and methods of frequency analysis such as Fourier and Huang-Hilbert Transforms.

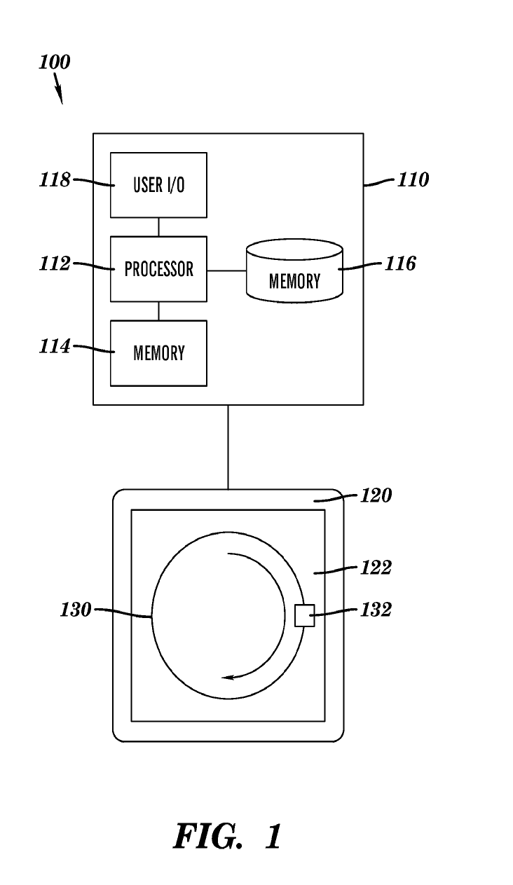

FIG. 1 shows a diagram of a system 100 according to the present invention. The system 100 can include a computer or data processing system 110 connected to a task monitoring device 120. The connection can be a wired or wireless connection (e.g, WiFi, Bluetooth, Zigbee, etc.). The computer system 110 can include one or more processors 112 and associated memory devices 114, 116 (e.g., volatile and non-volatile memory devices) and one or more user input and output devices 118 (e.g., display devices, keyboards, mice, etc.). The computer system 110 can also include software (e.g., operating systems and application programs) to facilitate the operation of the system and for receiving, storing and processing data. The task monitoring device 120 can take many forms and can include one or more sensors for recording subject motion data while the subject is performing a requested task. In accordance with one embodiment of the invention, the task monitoring device 120 can include a touch sensitive display screen 122 that can display objects 130, 132 and images on the screen and sense a subject making contact with the screen.

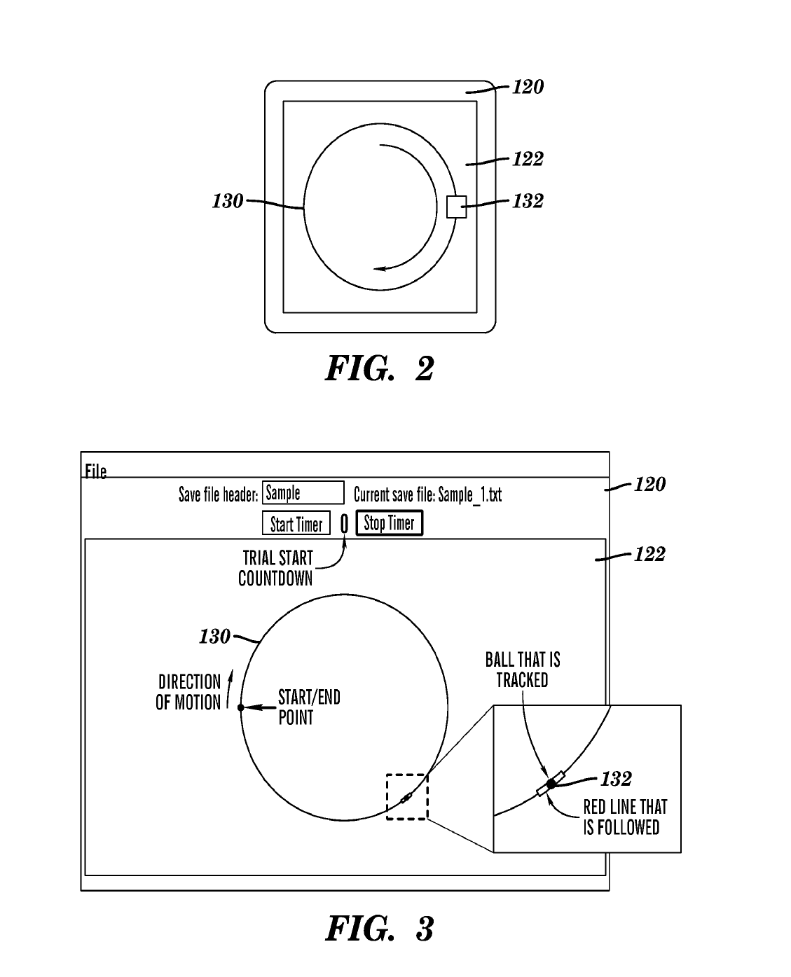

FIGS. 2 and 3 show diagrams of an example of a task according to some embodiments of the present invention. In accordance with the invention, a subject is provided with a task monitoring device 120 and asked to perform a task. In one embodiment, the subject is asked to place their finger (or a stylus) on an object 132 (e.g., a dot or a box) on the screen and maintain their finger in the center of the box as the box moves around the circle 130, as indicated by the arrow shown.

In accordance with the invention, one or more software application programs can be executed by the processor 112 to cause the circle and the object to appear on the screen. Other indicia (not shown), such as instructions and a count-down timer can be provided to assist the subject in performing the task. The subject can be instructed to perform the task of following the object around the circle and the task monitoring device 120 can sense the position of the subject's finger (or a pointing device, such as a stylus) and transfer this information to the computer system 110. The computer system 110, under software application control can record the position information along with time synchronization information. The subject can be asked to repeat the task (e.g., 4 revolutions around the circle) and/or perform the task in different directions and/or at different speeds. A sequence of tasks can be presented, for example, including different directions and/or different paths (e.g., circles, ovals, lines, polygons, spirals, etc.). In some embodiments, the path can move into or out of the screen (e.g., a driving simulation task). These tasks can be implemented under software application program control. In some embodiments, the data can be collected and processed in real-time in order to provide feedback to the user. For example, symbol, such as a box or a circle on the display can change colors to provide an indication of performance or size of the object can be enlarged or reduced as a function of performance.

The information and/or data collected can be used to produce a time series of data representing the task motion recorded. For example, the data recorded can represent the position of the subject's finger on the screen at predefined sampling intervals and time series representing the difference between the actual position and target position (e.g., the object, dot, or box on the path) can be determined. Next, the degree of complexity or irregularity can be quantified using an entropy measure, such as SampEn, for example, resulting in a Multiscale Entropy (MSE) plot of SampEn at various scale factors. In accordance with one embodiment of the invention, a Complexity Index (CI) can be determined as the area under the MSE curve for a predefined range of scale factors. The Complexity Index can be used as the neuromotor index (NI) or combined with other measures to form the neuromotor index.

In accordance with embodiments of the present invention, one or more of the NI values for a given subject can be stored and used to evaluate neuromotor function of the subject. An initial NI value can be used as a baseline from which to evaluate the subject to indicate the existence of disease or disability. During treatment and therapy, further subsequent evaluations in accordance with the invention can be compared with one or more prior evaluations (NI values) to assess the effectiveness of the treatment and/or therapy. In accordance with some embodiments of the invention, the NI indicates a level of complexity or adaptability of a subject given their state at the time of assessment. It is expected that with a healthy subject the complexity level for a given task will be higher than when the subject is fighting a disease or upon initially acquiring a disability. The efficacy of treatment or therapy can be assessed according to embodiments of the invention by comparing current NI values with prior NI values to determine whether current NI values are greater, indicating increased complexity and a return to healthy state.

In accordance with embodiments of the present invention, an integrative neuromotor index can be calculated as a function of one or more of the neuromotor performance signals or indices. These parameters can be directly combined through addition, by comparing a vector, or through implementation of a model. In one embodiment, this model can be developed using principal component analysis, support vector machines, neural networks, or other machine learning algorithm. Similar to the CI, the values for a given subject can be stored and used to evaluate neuromotor function of the subject.

In accordance with other embodiments of the invention, the tracking system can include a display (which could be a monitor or a projected image) and an eye-tracking system. In this embodiment, the task can include the subject following an object as it moves along a predefined or random path with their eyes. The eye tracking system can determine the location or position on the display of the subject's gaze over time. The gaze position sequence can be used as described herein to determine NI values.

In accordance with other embodiments of the invention, the tracking system can employ a motion capture system to obtain the body motion over time of the subject's entire body or elements of the subject's body (e.g., head, arms, hands, legs and feet). The body motion can be captured using well known motion caption devices and methods including, but not limited to, remote sensing devices such as cameras and reflective sensor (e.g., mm and high frequency sensing) and subject worn sensing devices, such as, accelerometers, gyroscopes, magnetometers, force sensitive resistors, inertial navigation devices, and combinations of remote sensing devices and body worn sensing devices.

In accordance with other embodiments of the invention, the tracking system can a light sensing target and the subject can move a pointing laser to trace the motion of the moving target. The target can be moved by a simple mechanism, such as a rotating disk or a more complex system, multi degree-of-freedom actuator, such as a robotic arm. The target can include one or more sensors that can be used to determine the position of the laser image (laser spot) on the target and the deviation of the image from a center or reference point on the target. The target can include an array of light sensors that become illuminated by laser image projected by the subject on the target. A time sequence of positions on the target can be used to track the movement and the residual signal can be determined as a function of the distance from a target point (e.g., a center point) of the target array and the brightest point (e.g. highest signal intensity) on the array illuminate by the subject. Alternatively a single sensor or array of sensors can be used and the residual signal can be determined as a function of signal intensity. The position to be tracked could also be on a display screen.

### Data Collection

The speed of the target object (e.g., a red line, a circle or a square, FIG. 3) around the circle was selected through testing, with the goal of achieving a speed of motion of the object that was slow enough to allow for microcontrol adaptations (error corrections), but not so slow that the subject would consciously stop and wait for the target to move. In accordance with one embodiment, a speed of 18 deg/sec around a circle with a 400 pixel diameter was selected. (The tablet used in this study had a screen size of 8.25×6.125 in, with a resolution of 125 ppi.) Other speeds can be selected based on the resolution of the position sensing device, in this example, the touch screen. The data collection software can output the coordinates for both the target and the subject's actual position. The sampling frequency can be 31.25 Hz, a value that was chosen taking into consideration the target speed and the pixel size. In order to measure micropauses where the velocity is zero, the sampling frequency and speed can be selected to detect motion from one sensing position to the next, with limited overlap, thus sampling too slow or having large pixels would result in an inaccurate detection of micropause duration.

### Data Processing

In accordance with some embodiments of the invention, a method and system for measuring a neuromotor tracking function can be provided in a way that is simple, accurate, quantitative and inexpensive. The outputs can include measurements referred to herein the neuromotor index (NI). NI can reflect a combination of features useful for a physiologic system to be adaptive. These features can include 1) correlations across multiple time scales, 2) accuracy, and 3) fluidity. For example, these features can be analogized to the properties that are universally understood to underlie great works of classical music. From a practical, clinical point of view, a measure of these features can be reflected in the neuromotor index by employing one or more methods designed to analyze series of data points (time series) derived from tracking or monitoring motion.

In accordance with some embodiments of the invention, the system or method can include measuring the mean and standard deviation of the error of patient position, the complexity index (CI) of the residual signal, the percentage of time within the designated region, and the number of micropauses. The task tracking system monitors the movement of the subject, records the raw values, and can determine a predicted age of the subject based on the measures and stored baseline measures. The value can also be compared to a previous baseline measure recording.

In some embodiments of the invention, the desired position of the user can be actually a region of a circle. If (x_{c}, y_{c}) are the coordinates of the center of the circle and (xₛ, yₛ) are the coordinates of the stylus at a recorded time point, then the instantaneous error at that time point can be defined as

Instantaneous Error=\sqrt{(xₛ−x_{c})²+(yₛ−y_{c})²}−r (1)

where r is the radius of the circle. The error time series is then the sequence of instantaneous errors or micro-errors. To account for initiation and termination effects, the first and last quarter or other portion of the circle can be removed. Thus, in this embodiment, the system can analyze, for example, a total of 3.5 revolutions out of the 4 revolutions collected. The mean and standard deviation of the residual can be calculated using standard methods.

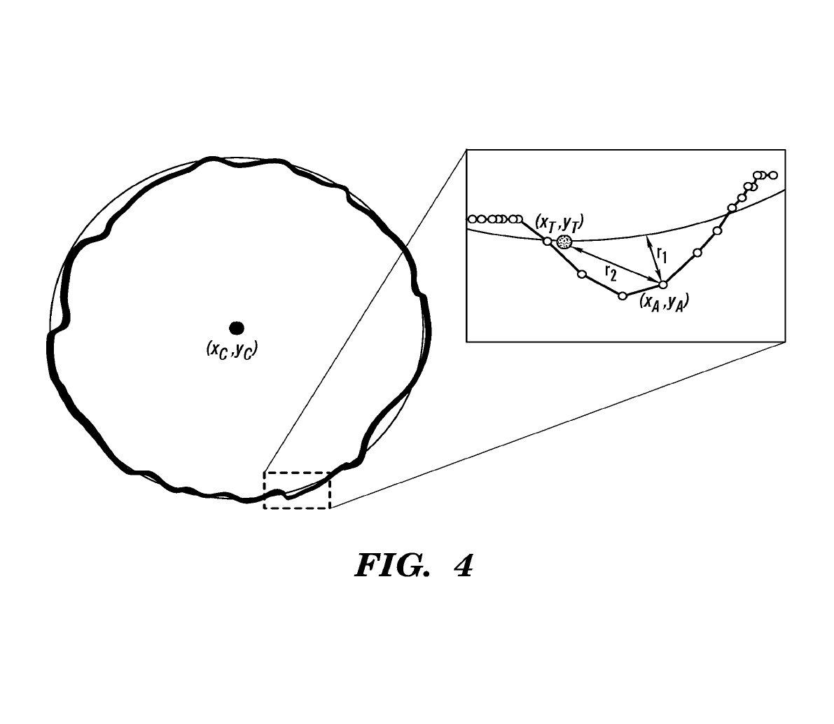

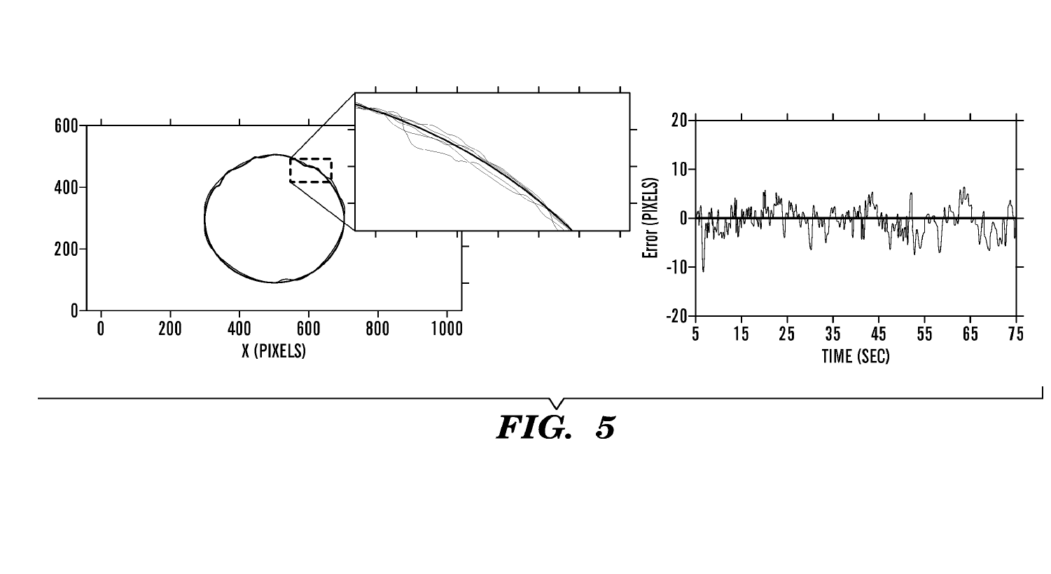

As shown in FIG. 4, in accordance with various embodiments of the invention, the error or residual values can be determined in one or more different ways. For example, the residual value r₁ can be determined as the difference between the actual stylus position (x_{A}, y_{A}) and the path being traced, which, for a circular path, extends along a line drawn between the actual stylus position (x_{A}, y_{A}) and the center of the circle (x_{C}, y_{C}). This residual value is independent of changes in speed. Alternatively, the residual value r₂ can be determined as the distance (e.g., Euclidean distance) between the actual stylus position (x_{A}, y_{A}) and the target position (x_{T}, y_{T}). For example, sqrt((x_{A}−x_{T})²+(y_{A}−y_{T})²). This residual value reflects changes in speed. In accordance with some embodiments of the invention, the residual signal can include a sequence of residual values determined at consecutive points in time. FIG. 5 shows a diagram of an example of a target path, an actual path and a residual signal.

This micro-error signal can also be calculated by determining the difference between the stylus position and the target region. In some embodiments of the invention, the micro-error signal can include position (angular or Cartesian coordinates) and/or velocity error. In other embodiments of the invention, the micro-error signal can include time error (e.g., the difference in time between actual arrival at a position and the expected arrival at a position).

The original MSE method was derived for the analysis of stationary time series. Since these tracking time series are highly non-stationary, the system and method according to the invention can use a detrending algorithm prior to calculating the CI values. For detrending, a moving average with a window of, for example, 21 points can be used. As described in detail elsewhere (Costa et al., 2005), the MSE comprises two steps: 1) deriving a set of coarse-grained time series that capture system dynamics over different time scales and 2) measuring the information content of each of the coarse-grained time series use sample entropy (SampEn). The MSE curves are the SampEn value plotted against the scale factor. The CI in one embodiment can be defined as the sum of the SampEn values for scales 1 through 4 based on the data collection rate, resolution of the tablet, and the total number of points recorded.

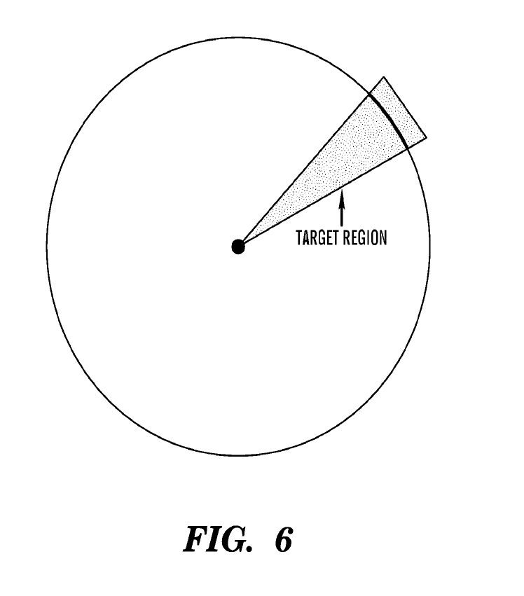

The target region can be defined at each of the generated time points by computing the rays from the circle origin to the boundaries of the moving target region, as shown in FIG. 6. If the cursor, represented by the ball, touches or falls within the target region, the time point can be considered within the region. The time increments associated with the pixels in the region can be summed to determine a total time in the region, as well as to verify the total task time. The percent time in the target region (PTTR) can be determined by dividing the total time in the region by the total task time.

Micropauses can be defined as occurring when the position of the stylus did not change between two consecutive time points, thus

Δ=|xᵢ₊₁−xᵢ|+|yᵢ₊₁−yᵢ|=0 (3)

where (x, y)ᵢ and (x, y)ᵢ₊₁ are the stylus coordinates at time i and i+1, respectively. The cumulative micropause duration is then the summation of the time increments associated with the repeated stylus coordinates. This parameter is limited only when the stylus location is not sampled frequently enough as pauses would be missed. If the data are sampled at higher rates, the cumulative micropause duration would not be affected.

In some embodiments of the invention, it may be appropriate to combine multiple parameters in the assessment of adaptability. These parameters may be combined in many ways, including machine learning algorithms (e.g. support vector machines, neural networks, hidden Markov models, etc.).

Additional embodiments for analyzing the correlations across multiple time scales and information content can be assessed with a variety of entropy-based analyses, including but not limited to: multiscale entropy (MSE), sample entropy (SampEn), approximate entropy (ApEn), Tsallis entropy, Kolmogorov entropy, and diffusion entropy. Each of these methodologies can be applied to the micro-error signal as defined previously.

Complementary techniques that measure correlation properties of time series are fractal and multifractal analyses, including those based on detrended fluctuation analysis, box-counting or wavelet analysis. These methods can be applied to the raw data or micro-error data. The multiscale entropy (MSE) method discussed in the preferred embodiment has certain attractive features for capturing correlations across time scales and information content in that it explicitly measures the entropy, not only of the original signal, but also of a family of signals derived therefrom, which represent multiple time scales. This technique allows one to distinguish highly variable signals without correlations (e.g., white noise) from more physiologic types of 1/f noise seen in the output of complex adaptive systems.

In additional embodiments, the accuracy of the tracking motions can be assessed with a number of measures in the time domain, including mean, standard deviation and higher moments of the histogram/probability distribution. These methods can be applied to the raw signal, micro-errors, velocity, acceleration, or other function of the raw signal.

Additional embodiments can assess the fluidity of motion by detecting oscillations that indicate the presence of a characteristic time scale associated with a pathologic process. For example, Parkinson's disease is associated with distinctive oscillations (tremor) at a frequency of around 5 Hz. These periodic dynamics can be detected and quantified using frequency domain analyses, including Fourier or wavelet-based methods, and methods based on the Hilbert-Huang Transform (HHT) and empirical mode decomposition (EMD). Each of these methods can be applied to the raw signal, micro-errors, velocity, acceleration, or other function of the raw signal. The assessment of fluidity (or lack thereof) of motion can also be assessed using measures of micropauses described previously. The greater the number of pauses and the longer their duration, the less fluid the motions are.

With these outcome measures, a model of the system can be developed using support vector machines (SVM) (Chang and Lin, 2001), which are a method of supervised learning used for classification. In order to develop a model, a set of training data with known classifications is required. Once trained, the model can be tested and used with different datasets. Here, a C-support vector classification formulation of the quadratic minimization problem (Chang and Lin, 2001; Boser et al., 1992; Cortes and Vapnik, 1995) with a radial basis function (RBF) can be used, implementing the “one-against-one” approach for multi-class classification (Chang and Lin, 2001; Knerr et al. 1990; Hsu and Lin, 2002), and a five-fold cross-validation model to minimize over-fitting the model. In accordance with one embodiment of the invention, training parameters include the tracing outcome measures for n-trials for the dominant hand, along with the corresponding subject gender. During the model development, there are two unknown parameters that must be solved, C, an error penalty parameter in the optimization, and γ, a RBF kernel parameter. The parameters C and γ can be determined, for example, by performing the cross-validation training optimization using a coarse grid search, then refine the search to obtain a better solution. The model can be trained with the best C and γ parameters. Using this model, the probability that a new data point falls within a particular age group can be estimated. Knowing a subjects' actual age, the system and method according to the invention can be used to assess whether their visuomotor skill falls above, below, or at their actual age.

The apparatus and method previously described can be implemented for the following applications:

Neurological assessment, including standard neurological exams and those in particularly affected groups such as the elderly, those with Parkinson's disease, multiple sclerosis, traumatic brain injury, micro traumatic brain injury, musculoskeletal injury, stroke, diabetes, cerebral palsy, etc. Baseline quantitative values can be determined from a sample healthy population and used as a threshold for detecting disease or disability. The data obtained from repeated assessments taken over time can be compared to determine how the patient progresses through healing, rehabilitation, training, therapy, etc. These measures can also be used to determine the efficacy of a drug dosage, or presence of side effects, for a particular diagnosis in providing an appropriate degree of motion adaptability.

Current concussion sideline assessment techniques use a very general scale that is affected by the person administrating the exam and the person responding to the questions. Methods and systems according to the present invention can provide a quantitative evaluation with appropriate resolution that is not limited by the subject interpretation of the person administering the test. For example, for athletic subject evaluation, such as in sports, evaluations according to the present invention can be given pre-season to establish a baseline and then after each game and/or potential injury, a more detailed understanding of the neuromotor pathways can be developed. Thus, if an athletic subject receives a concussion or other neuromotor injury, an evaluation according to the present invention can be used to determine the extent of the injury, the subject's progress through treatment and rehabilitation and when the subject has recovered back to their baseline measures. Similarly, embodiments of the present invention can be applied in other contexts, including, for example, military deployments, high speed activities, such as autoracing, and space travel. More generally, the evaluation according to the present invention can be performed during a standard physical and then can later be used for any person reporting head trauma.

Methods and systems according to embodiments of the invention can be used to develop a sobriety test. In accordance with embodiments of the invention, as a person increases their alcohol intake, their motion become less complex and less adaptable to perturbations, which can be quantitatively evaluated in accordance with the present invention. Given a baseline, evaluations according to the present invention can provide information on how able the person is to perform motor tasks.

Implantable neurological stimulators and implantable drug pumps continue to show promise in the treatment of a variety of diseases and ailments. Setting therapeutic levels and dosages is still difficult because it often relies on a clinician's observation of symptoms, or patient's self report of symptoms (such as tremor, etc.), during a dosage setting paradigm that can take hours, weeks, or months. Methods and systems according to embodiments of the present invention can be used quickly and precisely assess neuromotor adaptability and complexity would significantly improve the ability to tune these devices for individual needs.

Hydrocephalus, and its related disorders, involves the increase of cranial pressures from a build up of cerebral-spinal fluid around the brain. Typically excess fluid is normally drained to maintain cerebral pressure but in some cases this mechanism is faulty and cranial pressure can rise, leading to brain injury and neuromotor and cognitive dysfunction. Often the symptoms of an increase in cranial pressure are not apparent until the pressure reaches dangerous levels. Cerebral spinal shunts are often placed to drain excess fluid when the natural mechanisms fail but can clog and become nonfunctional over time. Assessment of cerebral pressure and shunt performance is typically invasive and expensive. Methods and systems according to embodiments of the present invention can be used to identify changes in neuromotor performance that indicate a dangerous change in pressure affecting neuromotor performance and cognition.

Research is ongoing in the field of electrical and mechanical assistance for improving pathologies associated with motor control. Methods and systems according to embodiments of the present invention can be used to provide information regarding the patient's neuromotor control with and without the assistive device. This additional information can be used for clinical assessment and evaluation of the efficacy of new assistive devices, including the objective assessment of the optimal range of parameters for a given individual.

A wide range of exercise and therapy protocols have been proposed for aging adults to enhance their motor control adaptability, including yoga, tai chi, strength training, etc. Methods and systems according to embodiments of the present invention can used to provide valuable quantitative information on the comparative efficacy of these exercise interventions in improving motor control adaptability.

During certain types of neurosurgery, the patient is kept awake and tested to confirm which parts of the brain are being affected. In accordance with embodiments of the invention, for example, eye-tracking based embodiment, can be used to monitor neuromotor ability, while still keeping the patient immobile.

In accordance with various embodiments of the present invention, emotion-based assessment, including assessment for depression treatment, post-traumatic stress disorder, combat fatigue, etc can be provided. With increased emotional stress, the body is less able to adapt, thus their motor tracking complexity should decrease. Methods and systems according to embodiments of the present invention can be used as a diagnostic to determine if a patient improves from a baseline measurement condition, or degrades when the baseline is during a neutral state.

Chronic or acute sleep deprivation can cause fatigue, decrease cognitive functionality, and decrease motor control. While drivers are not permitted to drive when under the influence of alcohol, there are no legal limitations on the permissibility of driving while fatigued. Methods and systems according to embodiments of the present invention can used to determine if a driver is alert and adaptable enough to drive. Embodiments of the invention can be incorporated into a car dash device or program. This embodiment might be especially useful for transit workers, rail works, long haul truck drivers, surgeons, and medical residents. Embodiments of the present invention can also be used for clinical assessment of the neuromotor pathway associated with fatigue and can be used to help with drug dosing.

Methods and systems according to embodiments of the present invention can also be used to analyze a robot performing similar tracking tasks. Embodiments of the present invention can be used to determine whether the robot is adaptable to perturbations in a similar manner as a healthy human. The present invention can be used to detect defective sensors, actuators and/or communication pathways.

In some applications, it may be appropriate to combine multiple parameters in the assessment of adaptability. These parameters may be combined in many ways, including machine learning algorithms (e.g. support vector machines, neural networks, hidden Markov models, etc.).

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

The following references are incorporated herein by reference in their entirety
1. Kessler R C, Berglund P, Demler O, Jin R, Koretz D, Merikangas K R et al. The epidemiology of major depressive disorder: results from the National Comorbidity Survey Replication (NCS-R). JAMA 2003; 289: 3095-3105.
2. Hasin D S, Goodwin R D, Stinson F S, Grant B F. Epidemiology of major depressive disorder: results from the National Epidemiologic Survey on Alcoholism and Related Conditions. Arch Gen Psychiatry 2005; 62: 1097-1106.
3. Murray C J, Lopez A D. Alternative projections of mortality and disability by cause 1990-2020: Global Burden of Disease Study. Lancet 1997; 349: 1498-1504.
4. Musselman D L, Evans D L, Nemeroff C B. The relationship of depression to cardiovascular disease. Arch Gen Psychiatry 1998; 55: 580-592.
5. Belmaker R H, Agam G. Major depressive disorder. N Engl J Med 2008; 358: 55-68
6. Penninx B W J H, Beekman A T F, Honig A, Deeg D J H, Schoevers R A, van Eijk J T M et al. Depression and cardiac mortality: results from a community-based longitudinal study. Arch Gen Psychiatry 2001; 58: 221-227.
7. Lesperance F, Frasure-Smith N, Talajic M, Bourassa M G. Five-year risk of cardiac mortality in relation to initial severity and one-year changes in depression symptoms after myocardial infarction. Circulation 2002; 105: 1049-1053.
8. Carney R M, Freedland K E, Miller G E, Jaffe A S. Depression as a risk factor for cardiac mortality and morbidity: a review of potential mechanisms. J Psychosom Res 2002; 53:897-902.
9. Parissis J T, Fountoulaki K, Filippatos G, Adamopoulos S, Paraskevaidis I, Kremastinos D. Depression in coronary artery disease: novel pathophysiologic mechanisms and therapeutic implications. Int J Cardiol 2007; 116: 153-160.
10. American Psychiatric Association. Diagnostic and Statistical Manual of Mental Disorders, 4th edn, DSM-IV-TR. (text revision). American Psychiatric Association: Washington D.C., 2000.
11. Steiger A, Kimura M. Wake and sleep EEG provide biomarkers in depression. J Psychiatr Res 2010; 44: 242-252.
12. Miller A H, Maletic V, Raison C L. Inflammation and its discontents: the role of cytokines in the pathophysiology of major depression. Biol Psychiatry 2009; 65: 732-741
13. Samuels A M. The brain-heart connection. Circulation 2007; 116: 77-84.
14. Valentini M, Parati G. Variables influencing heart rate. Prog Cardiovasc Dis 2009; 52: 11-19.
15. Costa M, Goldberger A L, Peng C-K. Multiscale entropy analysis of complex physiologic time series. Phys Rev Lett 2002; 89: 068102-1-068102-4.
16. Chialvo D R. Physiology: unhealthy surprises. Nature 2002; 419: 263.
17. Costa M, Goldberger A L, Peng C-K. Multiscale entropy analysis of biological signals. Phys Rev E Stat Nonlin Soft Matter Phys 2005; 71: 021906-1-18.
18. Yeragani V K. Major depression and long-term heart period variability. Depress Anxiety 2000; 12: 51-52.
19. Yeragani V K, Rao K A, Smitha M R, Pohl R B, Balon R, Srinivasan K. Diminished chaos of heart rate time series in patients with major depression. Biol Psychiatry 2002; 51: 733-744.
20. Glassman A H, Bigger J T, Gaffney M, Van Zyl L T. Heart rate variability in acute coronary syndrome patients with major depression: influence of sertraline and mood improvement. Arch Gen Psychiatry 2007; 64: 1025-1031.
21. Licht C M M, de Geus E J C, Zitman F G, Hoogendijk W J G, van Dyck R, Penninx B W J H. Association between major depressive disorder and heart rate variability in the Netherlands Study of Depression and Anxiety (NESDA). Arch Gen Psychiatry 2008; 65: 1358-1367.
22. Servant D, Logier R, Mouster Y, Goudemand M. Heart rate variability. Applications in psychiatry. Encephale 2009; 35: 423-428.
23. Kemp A H, Quintana D S, Gray M A, Felmingham K L, Brown K, Gatt J M. Impact of depression and antidepressant treatment on heart rate variability: a review and metaanalysis. Biol Psychiatry 2010; 67: 1067-1074.
24. Grassberger P. Information and Complexity Measures in Dynamical Systems. In: Atmanspacher H, Scheingraber H (eds). Information Dynamics. Plenum Press: New York, 1991 pp 15-33.
25. Goldberger A L, Giles F. Filley lecture. Complex systems. Proc Am Thorac Soc 2006; 3: 467-472.
26. Goldberger A L, Amaral L A, Hausdorff J M, Ivanov P Ch, Peng C-K, Stanley H E. Fractal dynamics in physiology: alterations with disease and aging. Proc Natl Acad Sci USA 2002; 19 (Suppl 1): 2466-2472.
27. Goldberger A, Peng C-K, Lipsitz L A. What is physiologic complexity and how does it change with aging and disease? Neurobiol Aging 2002; 23: 23-26.
28. Norris P R, Anderson S M, Jenkins J M, Williams A E, Morris Jr J A. Heart rate multiscale entropy at three hours predicts hospital mortality in 3,154 trauma patients. Shock 2008; 30: 17-22.
29. Riordan Jr W P, Norris P R, Jenkins J M, Morris Jr J A. Early loss of heart rate complexity predicts mortality regardless of mechanism, anatomic location, or severity of injury in 2178 trauma patients. J Surg Res 2009; 156: 283-289.
30. Ferrario M, Signorini M G, Magenes G. Complexity analysis of the fetal heart rate variability: early identification of severe intrauterine growth-restricted fetuses. Med Biol Eng Comput 2009; 47: 911-919.
31. Richman J S, Moorman J R. Physiological time series analysis using approximate entropy and sample entropy. Am J Physiol 2000; 278: H2039-H2049.
32. Pincus S M. Assessing serial irregularity and its implications for health. Ann NY Acad Sci 2001; 954: 245-267.
33. Task Force of the European Society of Cardiology the North American Society of Pacing Electrophysiology. Heart rate variability: standards of measurement, physiological interpretation, and clinical use. Circulation 1996; 93: 1043-1065.
34. Hamilton M. A rating scale for depression. J Neurol Neurosurg 1960; 23: 56-62.
35. Buysse D J, Reynolds C F, Monk T H, Berman S R, Kupfer D J. The Pittsburgh sleep quality index: a new instrument for psychiatric practice and research. Psychiatry Res 1989; 28: 193-213.
36. Leistedt S J-J, Coumans N, Dumont M, Lanquart J-P, Stam C J, Linkowski P. Altered sleep brain functional connectivity in acutely depressed patients. Hum Brain Mapp 2009; 30:2207-2219.
37. Agnew Jr H W, Webb W B, Williams R L. The first night effect: an EEG study of sleep. Psychophysiology 1966; 2: 263-266.
38. Rechtschaffen A, Kales A (eds) A manual of standardized terminology techniques and scoring system for sleep stages of human subjects. UCLA Brain Information Service/Brain Research Institute, University of California: Los Angeles, Calif., 1968.
39. Devlin P, Moody G, Mark R. Desing and evaluation of amodular real-time QRS detector. Proc 35th Ann Conf on Engineering in Medicine and Biology, Bethesda, USA; 1982; 40.
40. Mietus J E, Peng C K, Henry I, Goldsmith R L, Goldberger A L. The pNNx-files: re-examining a widely-used heart rate variability measure. Heart 2002; 88: 378-380.
41. Saul J P, Albrecht P, Berger R D, Cohen R J. Analysis of long term heart rate variability: meth-ods, 1/f scaling and implications. Comput Cardiol 1988; 14: 419-422.
42. Bigger J T, Steinman R C, Rolnitzky L M, Fleiss J L, Albrecht P, Cohen R J. Power law behavior of RR-interval variability in healthy middle-aged persons, patients with recent acute myocardial infarction, and patients with heart transplants. Circulation 1996; 93: 2142-2151.
43. Kerkhofs M, Linkowski P, Lucas F, Mendlewicz J. Twenty-four-hours patterns of sleep in depression. Sleep 1991; 14: 501-506.
44. Kupfer D J. Sleep research in depressive illness: clinical implications—a tasting menu. Biol Psychiatry 1995; 38: 391-403.
45. Schulz S, Koschke M, Bar K-J, Voss A. The altered complexity of cardiovascular regulation in depressed patients. Physiol Meas 2010; 31: 303-321.
46. Yang A C, Tsai S J, Yang C-H, Kuo C-H, Chen T-J, Hong C-J. Reduced physiologic complexity is associated with poor sleep in patients with major depression and primary insomnia. J Affect Disord, advanced online publication 2010 e-pub ahead of print.
47. Boettger S, Hoyer D, Falkenhahn K, Kaatz M, Yeragani V K, Bar K-J. Nonlinear broad band dynamics are less complex in major depression. Bipolar Disord 2008; 10: 276-284.
48. Jokinen V, Syvae\ddot{}nne M, Mae\ddot{} kikallio T H, Airaksinen K E J, Huikuri H V. Temporal agerelated changes in spectral, fractal and complexity characteristics of heart rate variability. Clin Physiol 2001; 21: 273-281.
49. Lipsitz L A. Aging as a Process of Complexity Loss. In: Deisboeck T S, Kresh J Y (eds). Topics in Biomedical Engineering International Book Series: Complex Systems Science in Biomedicine. Springer, 2006 pp 641-654.
50. Starr J M, McGurn B, Harris S E, Whalley L J, Deary I J, Shiels P G. Association between telomere length and heart disease in a narrow age cohort of older people. Exp Gerontol 2007; 42: 571-573.
51. Epel E S, Blackburn E H, Lin J, Dhabhar F S, Adler N E, Morrow J D et al. Accelerated telomere shortening in response to life stress. Proc Natl Acad Sci USA 2004; 101: 17312-17315.
52. Simon N M, Smoller J W, McNamara K L, Maser R S, Zalta A K, Pollack M H et al. Telomere shortening and mood disorders: preliminary support for a chronic stress model of accelerating aging. Biol Psychiatry 2006; 60: 432-435.
53. Mundt J C et al. Voice acoustic measures of depression severity and treatment response collected via interactive voice response (IVR) technology. Journal of Neurolinguistics 20 (2007) 50-64.
54. Pohl P, Winstein C, Fisher B. The locus of age-related movement slowing: Sensory processing in continuous goal-directed aiming. J Gerontol Psych Sci. 1996; 51B:94-102.
55. Boser B, Guyon I, Vapnik V. A training algorithm for optimal margin classifiers. In Proceedings of the Fifth Annual Workshop on Computational Learning Theory, pages 144-152. ACM Press, 1992.
56. Cortes C, Vapnik V. Support-vector network. Machine Learning, 20:273-297, 1995.
57. Chang C, Lin C. LIBSVM: a library for support vector machines, 2001. Software available at http://www.csie.ntu.edu.tw/˜cjlin/libsvm.
58. Costa M, Goldberger A, Peng C. Multiscale entropy analysis of complex physiologic time series. Phys Rev Lett 89(6):068102, 2002.
59. Costa M, Priplata A, Lipsitz L, Wu Z, Huang N, Goldberger A, Peng C. Noise and poise: Enhancement of postural complexity in the elderly with a stochastic resonance-based therapy. Europhys Lett 77:68008, 2007.
60. Davies A. The Influence of Age on Trail Making Test Performance. Journal of Clinical Psychology 24(1): 96-98, 1968.
61. Goldberger A, Amaral L, Hausdorff J, Ivanov P, Peng C, Stanley H. Fractal dynamics in physiology: alterations with disease and aging. Proc Natl Acad Sci USA. 2002 Feb. 19; 99 Suppl 1:2466-72.
62. Hocherman S, Alexandrovsky L, Badarny S, Honigman S. L-DOPA improves visuo-motor coordination in stable Parkinson's disease patients. Parkinsonism & Related Disorders 4(3): 129-136, 1998.
63. Hsu C, Lin C. A comparison of methods for multi-class support vector machines. IEEE Transactions on Neural Networks, 13(2):415-425, 2002.
64. Inzelberg R, Schechtman E, Hocherman S. Visuo-Motor Coordination Deficits and Motor Impairments in Parkinson's Disease. PLoS ONE 3(11) e3663, 2008.
65. Knerr S, Personnaz L, Dreyfus G. Single-layer learning revisited: a stepwise procedure for building and training a neural network. In J. Fogelman, editor, Neurocomputing: Algorithms, Architectures and Applications. Springer-Verlag, 1990.
66. Krebs H, Aisen M, Volpe B, Hogan N. Quantization of continuous arm movements in humans with brain injury. Proceedings of the National Academy of Sciences of the United States of America 96(8): 4645-4649, 1999.
67. Leistedt S J-J, Linkowski P, Lanquart J-P, Mietus J E, Davis R B, Goldberger A L, Costa M D. Decreased Neuroautonomic Complexity in Men During an Acute Major Depressive Episode: Analysis of Heart Rate Dynamics. Transl Psychiatry (2011) 1, e27, doi:10.1038/tp.2011.23.
68. Meyer D, Smith J, Kornblum S, Abrams R, Wright C. Speed accuracy tradeoff to movements: toward a theory of rapid voluntary action. In Attention and Performance XIII: Motor Representation and Control, M. Jeannerod, ed. L.E.A. Publishers, Hillsdale, N.J., pp. 173-226, 1990.
69. Rohrer B, Fasoli S, Krebs H, Hughes R, Volpe B, Frontera W, Stein J, Hogan N. Movement Smoothness Changes during Stroke Recovery. The Journal of Neuroscience 22(18): 8297-8304, 2002.
70. Tirosh E, Perets-Dubrovsky S, Davidovitch M, Hocherman S. Visuomotor Tracking Related to Attention-Deficit Hyperactivity Disorder (ADHD). Journal of Child Neurology 21(6): 502-507, 2006.
71. Tombaugh T. Trail Making Test A and B: Normative data stratified by age and education. Archives of Clinical Neuropsychology 19(2): 203-214, 2004.
72. von Hofsten C. Predictive reaching for moving objects by human infants. Journal of Experimental Child Psychology 30(3): 369-382, 1980.
73. U.S. Pat. No. 7,509,161 to Viertio-Oja.
74. U.S. Pat. No. 6,631,291 to Viertio-Oja, et al.
75. Fitts P. The information capacity of the human motor system in controlling the amplitude of movement. J Exp Psychol. 1954; 47:381-391.
76. Lipsitz L, Goldberger A. Loss of ‘complexity’ and aging: Potential applications of fractals and chaos theory to senescence. JAMA. 1992; 267:1806-1809.
77. Goldberger A, Amaral L, Hausdorff J, Ivanov P, Peng C, Stanley H. Fractal dynamics in physiology: Alterations with disease and aging. Proc Natl Acad Sci USA. 2002; 99 Suppl 1:2466-72.
78. Krampe R. Aging, expertise, and fine motor movement. Neurosci Biobehav R. 2002; 26: 769-776.
79. Goldberger A. Non-linear dynamics for clinicians: chaos theory, fractals, and complexity at the bedside. Lancet. 1996; 347:1312-1314.
80. Newell K M, Broderick M P, Deutsch K M, Slifkin A B. Task Goals and Change in Dynamical Degrees of Freedom with Motor Learning. J Experimental Psychology 29(2):379-387, 2003.
81. Slifkin A B, Vaillancourt D E, Newell K M. Intermittency in the Control of Continuous Force Production. J Neurophysiology 84: 1708-1718, 2000.
82. Vaillancourt D E, Newell K M. Changing complexity in human behavior and physiology through aging and disease. Neurobiology of Aging 23: 1-11, 2002.

1. A method for assessing neurologic function comprising:
providing a tracking device for tracking movement of a subject performing a predefined task, the tracking device producing a signal representative of the movements of the subject;
determining a residual signal as a function of movement according to the predefined task and the signal representative of the movements of the subject;
determining a neuromotor index as a function of the residual signal; and
storing the neuromotor index in a memory.

2. The method according to claim 1 wherein the residual signal is determined as function of a difference between an expected position corresponding to the predefined defined task and an actual position from the signal representative of the movements of the subject.

3. The method according to claim 1 wherein determining the neuromotor index includes determining an entropy value over multiple time scales as a function of the residual signal.

4. The method according to claim 1 further comprising:
determining from the signal representative of the movements of the subject at least one time interval during which there is no movement of the subject;
determining a micropause index as a function of a sum of at least one time interval during which there is no movement of the subject; and
determining the neuromotor index as a function of the residual signal and the micropause index.

5. The method according to claim 1 further comprising:
determining from the signal representative of the movements of the subject, movements of the subject that correspond to at least one region in space and a summation of a total time within the region; and
determining a percentage time in target region index as a function of the summation of the total time with the region and total task time; and
determining the neuromotor index as a function of the residual signal and the percentage time in target region index.

6. The method according to claim 5 further comprising:
determining from the signal representative of the movements of the subject at least one time interval during which there is no movement of the subject; and
determining a micropause index as a function of a sum of at least one time interval during which there is no movement of the subject; and
determining the neuromotor index as a function of the residual signal, the micropause index and the percentage time in target region index.

7. The method according to claim 1 further comprising comparing the neuromotor index to a baseline neuromotor index.

8. The method according to claim 7 wherein the baseline neuromotor index is a baseline neuromotor index determined for a sample population similar to the subject.

9. The method according to claim 7 wherein the baseline neuromotor index is a prior neuromotor index determined for subject at prior point in time.

10. The method according to claim 7 wherein the baseline neuromotor index is a neuromotor index determined for a sample population similar to the subject.

11. The method according to claim 1 wherein the tracking device tracks the movements of a subject tracing an object moving along a path.

12. The method according to claim 11 wherein the path is a circular path.

13. The method according to claim 1 wherein the tracking device tracks the movements of a subject's eyes while the subject follows an object moving along a path.

14. The method according to claim 1 wherein the tracking device tracks a position of a laser image on a target object as the subject moves a laser to follow the object as it moves along a path.

15. A system for assessing neurologic function comprising:
a tracking device for tracking movement of a subject performing a predefined task, the tracking device producing a signal representative of the movements of the subject;
a computer system including a computer processor and associated memory, the computer system being connected to the tracking device and receiving the signal representative of the movements of the subject,
the computer system including a residual module adapted to determine a residual signal as a function of movement according to the predefined task and the signal representative of the movements of the subject, an index module adapted to determine a neuromotor index as a function of the residual signal, and a storage module adapted to store the neuromotor index in a memory.

16. The system according to claim 15 wherein the residual module is adapted to determine the residual signal as function of a difference between an expected position corresponding to the predefined defined task and an actual position from the signal representative of the movements of the subject.

17. The system according to claim 15 wherein the index module is adapted to determine the neuromotor index by determining an entropy value over multiple time scales as a function of the residual signal.

18. The system according to claim 15 wherein the computer system further includes a micropause module adapted to determine from the signal representative of the movements of the subject at least one time interval during which there is no movement of the subject and to determine a micropause index as a function of a sum of at least one time interval during which there is no movement of the subject; and
wherein the index module is adapted to determine the neuromotor index as a function of the residual signal and the micropause index.

19. The system according to claim 15 wherein the computer system further includes a percentage time module adapted to determine from the signal representative of the movements of the subject, movements of the subject that correspond to at least one region in space and a summation of a total time within the region and to determine a percentage time in target region index as a function of the summation of the total time with the region and total task time; and
wherein the index module is adapted to determine the neuromotor index as a function of the residual signal and the percentage time in target region index.

20. The system according to claim 19 wherein the computer system further includes a micropause module adapted to determine from the signal representative of the movements of the subject at least one time interval during which there is no movement of the subject and to determine a micropause index as a function of a sum of at least one time interval during which there is no movement of the subject; and
wherein the index module is adapted to determine the neuromotor index as a function of the residual signal, the micropause index and the percentage time in target region index.

21. The system according to claim 1 wherein the computer system further includes a comparison module adapted to compare the neuromotor index to a baseline neuromotor index.

22. The system according to claim 21 wherein the baseline neuromotor index is a baseline neuromotor index determined for a sample population similar to the subject.

23. The system according to claim 21 wherein the baseline neuromotor index is a prior neuromotor index determined for subject at prior point in time.

24. The system according to claim 21 wherein the baseline neuromotor index is a neuromotor index determined for a sample population similar to the subject.

25. The system according to claim 15 wherein the tracking device tracks the movements of a subject tracing an object moving along a path.

26. The system according to claim 25 wherein the tracking device includes a touch screen and the signal representative of the movements of the subject is determined from input from the touch screen.

27. The system according to claim 25 wherein the path is a circular path.

28. The system according to claim 15 wherein the tracking device includes an eye tracking system that tracks the movements of a subject's eyes while the subject follows an object moving along a path.

29. The system according to claim 15 wherein the tracking device includes an optical sensor that tracks a position of a laser image on a target object as the subject moves a laser to follow the object as it moves along a path.
